(19) United States
(12) Patent Application Publication
(10) Pub. No.: US 2010/0114165 A1
(43) Pub. Date: May 6, 2010

(54) POSTERIOR DYNAMIC STABILIZATION SYSTEM WITH PIVOTING COLLARS

(75) Inventor: Kameron Scott Ely, Cedar Park, TX (US)

Correspondence Address: SPRINKLE IP LAW GROUP, 1301 W. 25TH STREET, SUITE 408, AUSTIN, TX, 78705, US

(73) Assignee: Abbott Spine, Inc., Austin, TX (US)

(21) Appl. No.: 12/264,752

(22) Filed: Nov. 4, 2008

(51) Int. Cl. A61B 17/70; A61B 17/04

(52) U.S. Cl. 606/246; 606/254; 606/264; 606/278; 606/301

(57) ABSTRACT

A dynamic spinal stabilization system. The system can include a spinal stabilization rod and a pivoting collar which can be attached to a vertebra. The pivoting collar can define a bore which has internal surfaces and corresponding contact points. The bore can accept the rod and allow it to pivot about a contact point. The internal surfaces can be structured, shaped, and dimensioned to limit the range of motion of the rod in some or all directions. One portion of the rod can be flexible while another portion of the rod can be flexible. The flexible portion of the rod can engage the pivoting collar and bend within the pivoting collar to provide a greater range of motion.

### TECHNICAL FIELD

This disclosure relates generally to spinal stabilization systems, and more particularly to spinal implants for dynamically stabilizing human spines. Even more particularly, this disclosure relates to embodiments of a pivoting collar and a posterior dynamic stabilization system utilizing the same.

### BACKGROUND

The human spine consists of segments known as vertebrae separated by intervertebral disks 28 and held together by various ligaments. There are 24 movable vertebrae—7 cervical, 12 thoracic, and 5 lumbar. Each of the movable vertebrae has a somewhat cylindrical bony body (often referred to as the centrum), a number of winglike projections, and a bony arch. The bodies of the vertebrae form the supporting column of the skeleton. The arches of the vertebrae are positioned so that the spaces they enclose form a curvilinear passage which is often referred to as the vertebral canal. The vertebral canal houses and protects the spinal cord (which includes bundles of sensory and motor nerves for sensing conditions in or affecting the body and commanding movements of various muscles). Within the vertebral canal, spinal fluid can circulate to cushion the spinal cord and carry immunological cells to it, thereby protecting the sensory and motors nerves therein from mechanical damage and disease. Ligaments and muscles are attached to various projections of the vertebrae such as the superior-inferior, transverse, and spinal processes. Other projections, such as vertebral facets, join adjacent vertebrae to each other, in conjunction with various attached muscles, tendons, etc. while still allowing the vertebrae to move relative to each other.

Spines may be subject to abnormal curvature, injury, infections, tumor formation, arthritic disorders, punctures of the intervertebral disks, slippage of the intervertebral disks from between the vertebrae, or combinations thereof. Injury or illness, such as spinal stenosis and prolapsed disks may result in intervertebral disks having a reduced disk height, which may lead to pain, loss of functionality, reduced range of motion, disfigurement, and the like. Scoliosis is one relatively common disease which affects the spinal column. It involves moderate to severe lateral curvature of the spine and, if not treated, may lead to serious deformities later in life. Such deformities can cause discomfort and pain to the person affected by the deformity. In some cases, various deformities can interfere with normal bodily functions. For instance, some spinal deformities can cause the affected person's rib cage to interfere with movements of the respiratory diaphragm, thereby making respiration difficult. Additionally, some spinal deformities noticeably alter the posture, gate, appearance, etc. of the affected person, thereby causing both discomfort and embarrassment to those so affected. One treatment involves surgically implanting devices to correct such deformities, to prevent further degradation, and to mitigate symptoms associated with the conditions which may be affecting the spine.

Modern spine surgery often involves spinal stabilization through the use of spinal implants or stabilization systems to correct or treat various spine disorders and/or to support the spine. Spinal implants may help, for example, to stabilize the spine, correct deformities of the spine, facilitate fusion of vertebrae, or treat spinal fractures and other spinal injuries. Spinal implants can alleviate much of the discomfort, pain, physiological difficulties, embarrassment, etc. that may be associated with spinal deformities, diseases, injury, etc.

Spinal stabilization systems typically include corrective spinal instrumentation that is attached to selected vertebra of the spine by bone anchors, screws, hooks, clamps, and other implants hereinafter referred to as “bone anchors.” Some corrective spinal instrumentation includes spinal stabilization rods, spinal stabilization plates that are generally parallel to the patient's back, or combinations thereof. In some situations, corrective spinal instrumentation may also include superior-inferior connecting rods that extend between bone anchors (or other attachment instrumentation) attached to various vertebrae along the affected portion of the spine and, in some situations, adjacent vertebrae or adjacent boney structures (for instance, the occipital bone of the cranium or the coccyx). Spinal stabilization systems can be used to correct problems in the cervical, thoracic, and lumbar portions of the spine, and are often installed posterior to the spine on opposite sides of the spinous process and adjacent to the superior-inferior process. Some implants can be implanted anterior to the spine and some implants can be implanted at other locations as selected by surgical personnel such as at posterior locations on the vertebra.

Often, spinal stabilization may include rigid support for the affected regions of the spine. Such systems can limit movement in the affected regions in virtually all directions. Such spinal stabilizations are often referred to as “static” stabilization systems and can be used in conjunction with techniques intended to promote fusion of adjacent vertebrae in which the boney tissue of the vertebrae grow together, merge, and assist with immobilizing one or more intervertebral joints. More recently, so called “dynamic” spinal stabilization systems have been introduced wherein the implants allow at least some movement (e.g., flexion or extension) of the affected regions of the spine in at least some directions.

Dynamic stabilization systems therefore allow the patient greater freedom of motion at the treated intervertebral joint(s) and, in some cases, improved quality of life over that offered by static stabilization systems.

### SUMMARY

In one embodiment, a system for dynamically stabilizing a portion of a spine is provided. The system can include a spinal stabilization rod and a collar which can be attached to one of the vertebrae of the spine. The collar can define a bore, an internal surface of the bore, and a contact point on the internal surface. The bore can be shaped and dimensioned to accept the spinal stabilization rod and to allow the spinal stabilization rod to pivot about the contact point. In some embodiments, the spinal stabilization rod can be flexible so that it can bend about the contact point.

Regarding the bore, various embodiments include internal surfaces of differing shapes including, in some embodiments, generally semi-spherical internal surfaces. The internal surface can be further shaped and configured to limit the range through which the spinal stabilization rod pivots. For instance, the internal surface can limit the spinal stabilization rod to a range of about six degrees in any direction. In some embodiments, the range through which the spinal stabilization rod can pivot can differ for differing directions. In some embodiments, at least a portion of the bore can have an oval cross sectional shape.

In some embodiments, the system can include a second collar. Some second collars can define a slot for accepting the spinal stabilization rod. The slot of the second collar can have a diameter which is larger than the smallest diameter of the bore. Regarding the spinal stabilization rod, it can have two portions one of which has a diameter corresponding to that of the slot of the second collar. The other portion of the spinal stabilization rod can have a diameter corresponding to the smallest diameter of the bore. In some embodiments, the spinal stabilization rod can include a transition portion between the first and the second portions.

One embodiment provides a collar for dynamically stabilizing a portion of a spine in conjunction with a spinal stabilization rod. The collar can include a body which defines a bore, an internal surface of the bore, and a contact point on the internal surface. The bore can be shaped and dimensioned to accept the spinal stabilization rod and to allow the spinal stabilization rod to pivot about the contact point. In some embodiments, the spinal stabilization rod can be flexible so that it can bend about the contact point.

Regarding the bore, various embodiments include internal surfaces of differing shapes including, in some embodiments, generally semi-spherical internal surfaces. The internal surface can be further shaped and configured to limit the range through which the spinal stabilization rod pivots. For instance, the internal surface can limit the spinal stabilization rod to a range of about six degrees in any direction. In some embodiments, the range through which the spinal stabilization rod can pivot can differ for differing directions. In some embodiments, at least a portion of the bore can have an oval cross sectional shape.

Embodiments provide spinal stabilization systems which can statically stabilize two or more vertebrae while dynamically stabilizing one or more other vertebrae.

In some embodiments, spinal stabilization systems can allow rotation of certain vertebrae about one or more axes thereby allowing patients to flex/extend, rotate, or bend (or combinations thereof) various portions of their back. Thus, spinal stabilization systems of various embodiments can allow patients to bend or arch their backs, twist their torsos, bend side-to-side, and combinations thereof. Furthermore, embodiments provide dynamic stabilization systems which require no closure member or other components besides a spinal stabilization collar and rod.

These, and other, aspects will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the disclosure, and the disclosure includes all such substitutions, modifications, additions, or rearrangements.

### BRIEF DESCRIPTION OF THE DRAWINGS

A more complete understanding of the disclosure and the advantages thereof may be acquired by referring to the following description, taken in conjunction with the accompanying drawings in which like reference numbers generally indicate like features.

FIG. 1 depicts a graphical representation of a spinal stabilization patient.

FIG. 2 depicts a graphical representation of a human spine.

FIG. 3 depicts a simplified top view of one embodiment of a posterior spinal dynamic stabilization system installed with a pair of pivoting collars.

FIG. 4 depicts a simplified side view of one embodiment of a posterior spinal dynamic stabilization system in which a pivoting collar is coupled to a flexible portion of a spinal stabilization rod.

FIG. 5 depicts a simplified cross-sectional view of one embodiment of a spinal stabilization system attached to a first vertebra via a first bone screw having a clamping collar and to a second vertebra via a second bone screw having a pivoting collar.

FIG. 6 depicts a simplified cross-sectional view of one embodiment of a spinal stabilization system, illustrating a kinematical interaction between one embodiment of a spinal stabilization rod and a pivoting collar.

FIG. 7 depicts a cross-sectional view of one embodiment of a pivoting collar.

FIG. 8 depicts one embodiment of a bore of a pivoting collar, providing selected limits to the range of motion of a spinal stabilization rod in differing directions.

FIG. 9 depicts a cross-sectional view of one embodiment of a pivoting collar.

### DETAILED DESCRIPTION

The disclosure and the various features and advantageous details thereof are explained more fully with reference to the non-limiting embodiments detailed in the following description. Descriptions of well known starting materials, manufacturing techniques, components and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

As used herein, the terms “comprises,” “comprising,” “includes,” “including,” “has,” “having” or any other variation thereof, are intended to cover a non-exclusive inclusion. For example, a process, process, article, or apparatus that comprises a list of elements is not necessarily limited only those elements but may include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example”, “for instance”, “e.g.”, “in one embodiment”.

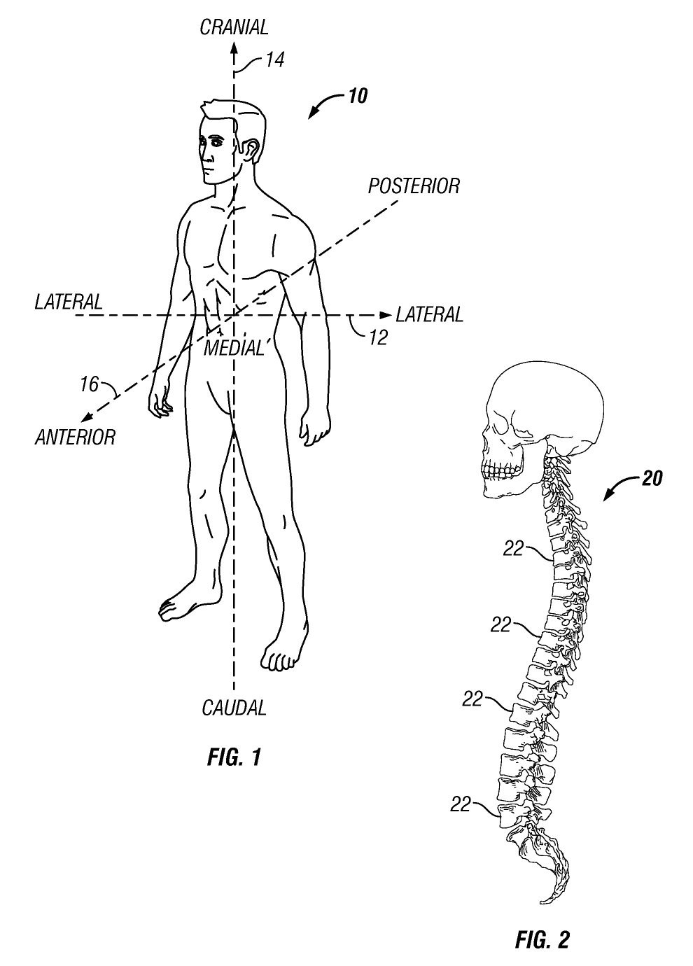

FIG. 1 depicts a graphical representation of spinal stabilization patient 10. Patient 10 generally possesses the capability to move according to many degrees of freedom which are at least partially defined with reference to medial-lateral axis 12, cranial-caudal axis 14, and anterior-posterior axis 16. More particularly, patients 10 may move such that portions of their backs (e.g., vertebrae) flex or extend (i.e., rotate about axes generally parallel to medial-lateral axis 12). For instance, patients 10 might lean forward or arch their backs. Patients 10 may also move such that portions of their backs rotate about axes generally parallel to cranial-caudal axis 14. For instance, patients 10 might twist their torsos to look behind themselves. Furthermore, patients 10 might bend to one side (or the other) thereby causing portions of their backs to rotate about axes parallel to anterior-posterior axis 16. Moreover, patients 10 might move such that portions of their backs translate relative to other portions of patients' 10 backs along axes 12, 14, or 16. It is also likely that movements of patients 10 will involve various combinations of the aforementioned degrees of freedom.

FIG. 2 depicts a human axial skeleton including a skull (composed of numerous cranial bones (such as parietal bones, temporal bones, zygomatic bones, mastoid bones, maxilla bones, mandible bones, etc.) and spine 20 including numerous vertebrae 22, intervertebral disks, etc. As discussed previously, spine 20 carries loads imposed on the patient's body and generated by patient 10. Vertebrae 22 cooperate to allow spine 20 to extend, flex, rotate, etc. (as discussed with reference to FIG. 1) under the influence of various muscles, tendons, ligaments, etc. attached to spine 20. Spine 20 can also cooperate with various muscles, tendons, ligaments, etc. to cause other anatomical features of the patient's body to move. However, certain conditions can cause damage to spine 20, vertebrae 22, intervertebral disks, etc. and can impede the ability of spine 20 to move in various manners. These conditions include, but are not limited to abnormal curvature, injury, infections, tumor formation, arthritic disorders, puncture, or slippage of the intervertebral disks, and injuries or illness such as spinal stenosis and prolapsed disks As some of these conditions progress, or come into existence, various symptoms can indicate the desirability of stabilizing spine 20 or portions thereof. As a result of various conditions, the ability of patient 10 to move, with or without pain or discomfort, can be impeded. Based on such indications, medical personnel can recommend attaching one or more spinal stabilization systems to vertebrae 22 among other remedial actions such as physical therapy.

It may be helpful at this juncture to briefly describe portions of vertebrae 22. Spinous processes and transverse processes allow tendons, muscles, etc. to attach to spine 20 for movement of spine 20 and various anatomical structures which are attached to spine 20 or affected thereby in various manners. These anatomical structures can include the patient's ribs, hips, shoulders, head, legs, etc. Spinous processes extend generally in a posterior and slightly inferior direction from vertebrae 22. Transverse processes also extend generally laterally from vertebrae 22 and allow muscles and tendons to attach to vertebra 22. Vertebral facets join adjacent vertebrae 22 to each other while allowing motion there between by being in sliding contact with corresponding vertebral facets of these adjacent vertebrae 22. During certain types of motion of spine 20 (such as flexing and extending) caused (or resisted) by various muscles, vertebrae 22 tend to rotate relative to each other about axes of rotation generally in the vertebral bodies (and more particularly proximal to points about one third of the anterior-posterior length of the vertebral bodies away from the posterior surface of these vertebral bodies). Since vertebral facets allow vertebrae 22 to articulate about these axes of rotation, no, or little, reactionary forces or moments are generated by healthy spines 20 themselves during ordinary movements.

Previously available approaches to dynamically stabilizing spine 20 include attaching stabilization rods to spine 20 in manners causing the rods to lie posterior to the spinous processes and therefore anatomically distant from intravertebral areas in which the vertebral axes of rotation lie. Since such previously available stabilization rods are distant from the vertebral axes of rotation they tend to generate reaction forces which resist movement of spine 20. Thus, as spine 20 extends or flexes, these previously available stabilization rods impede movement of spine 20. More particularly, the distances between vertebral axes of rotation can act as moment arms thereby generating moments and forces on spine 20. Therefore, spine 20 can cause reaction forces on the previously available spinal stabilization systems that can degrade the mechanical integrity and functioning of such spinal stabilization systems. Moreover, because such moments and forces (or their reactions) act on spine 20, spine 20 and patient 10 comfort and health can be adversely affected. As a result, the range of motion and patient comfort could be adversely affected with previously available spinal stabilization approaches. In addition, the moments and forces generated due to the anatomically significant distances between the vertebral axes of rotation and the previously available spinal stabilization systems can degrade the mechanical integrity of and functioning of such spinal stabilization systems.

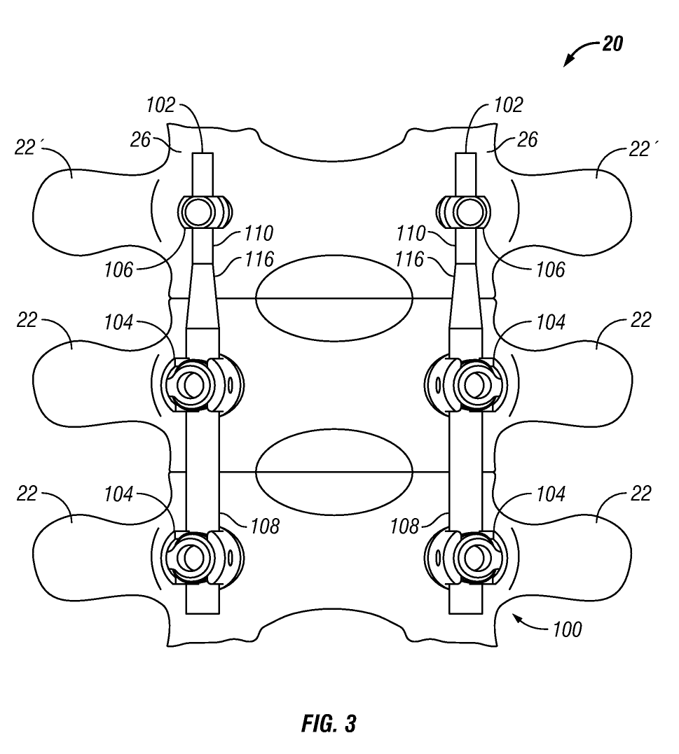

FIG. 3 depicts one embodiment of spinal stabilization system 100. Spinal stabilization system 100 includes at least one spinal stabilization rod 102 and one or more clamping collars 104 and pivoting collars 106. More particularly, as shown in FIG. 3, spinal stabilization system 100 can include a pair of spinal stabilization rods 102, two pairs of clamping collars 104, and a pair of pivoting collars 106. Pairs of clamping collars 104 can be attached to vertebrae 22 on the opposite sides of spinous process by bone screws, anchors, wires, etc. as can pairs of pivoting collars 106. Spinal stabilization rods 102 can be positioned on opposite sides of the vertebral spinous processes as shown. Furthermore, clamping collars 104 can securely clamp spinal stabilization rods 102 so that clamping collars 104 hold adjacent pairs of vertebrae 22 in fixed relationship to each other as shown by FIG. 3. Thus, spinal stabilization system 100 can statically stabilize these particular vertebrae 22. Over time, these particular statically stabilized vertebrae 22 may grow together to form one boney mass thereby fusing and permanently stabilizing these vertebrae 22.

However, as indicated by some patient 10 conditions, it may be desirable to dynamically stabilize some other particular vertebra 22′ with respect to other vertebrae 22. For instance, medical personnel may deem it desirable to allow vertebra 22′ to translate relative to other vertebrae 22 while also allowing selected amounts of rotation of vertebra 22′. For instance, surgical personnel may deem it desirable that vertebra 22′ be allowed to rotate relative to one or more axis 12, 14, or 16 (see FIG. 1). In such situations, among others, medical personnel may attach pivoting collars 106 to vertebra 22′ and to engage pivoting collar 106 with spinal stabilization rod 102.

More specifically, medical personnel may select spinal stabilization rod 102 which includes rigid portion 108 and flexible portion 110. Rigid portion 108 can be of a material, shape, and dimension sufficient to withstand various loads (forces, moments, torques, etc.) expected to be applied to vertebrae 22. Flexible portion 110 can be of a material, shape, and dimensions to withstand selected loads on vertebra 22′ (and adjacent vertebrae 22) while allowing relatively unrestricted motion in response to (or to generate) other loads. Flexible portions 110 of spinal stabilization rods 102 can pivotably and slidably engage pivoting collars 106 as discussed herein.

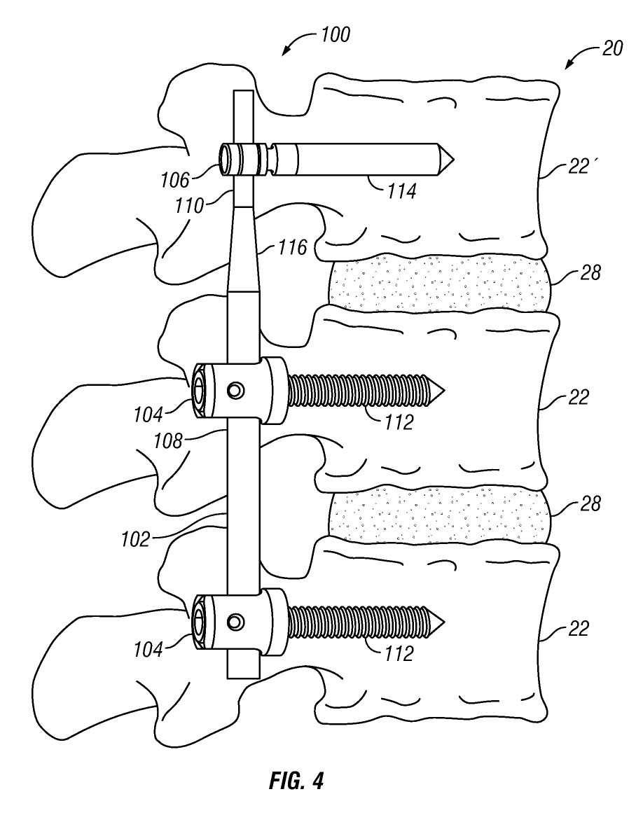

With reference now to FIG. 4, one embodiment of spinal stabilization system 100 is illustrated. More specifically, FIG. 4 illustrates spine 20 with vertebra 22 and 22′ and intervertebral disks 28 along with the following components of spinal stabilization system 100: spinal stabilization rod 102, clamping collars 104, pivoting collar 106, and bone screws 112 and 114. Bone screws 112 and 114 can attach collars 104 and 106 to vertebrae 22 and 22′ respectively. Bone screws 112 can be made of materials and have shapes and dimensions sufficient to withstand loads expected to be imposed thereon. In some embodiments, loads experienced by bone screws 114 can be less than those experienced by bone screws 112 since pivoting collars 106 might not constrain spinal stabilization rod 102 in as many directions as clamping collars 104. Bone screws 114, which attach pivoting collar 106 to vertebra 22′ can be made of materials and have shapes and dimensions sufficient to withstand loads expected to be imposed thereon. While FIG. 4 illustrates bone screws 112 and 114 attaching collars 104 and 106 to vertebrae 22 and 22′ those skilled in the art will understand that other types of attachment devices can be used in conjunction with collars 104 and 106.

Moreover, FIG. 4 illustrates that rigid portion 108 of spinal stabilization rod 102, can engage, and be securely clamped by, clamping collars 104. FIG. 4 also illustrates that flexible portion 110 of spinal stabilization rod 102 can pivotably and slidably engage pivoting collar 106. Thus, FIG. 4 illustrates that certain vertebrae 22 can be statically stabilized while other vertebrae 22 and 22′ can be dynamically stabilized. For illustrative purposes, FIG. 4 also shows that spinal stabilization rod 102 can be in some reference position which, in FIG. 4, is shown as being generally straight through collars 104 and 106. However, it is understood that other reference positions for spinal stabilization rod 102 are possible and within the scope of the disclosure. For instance, spinal stabilization rod 102 might have certain portions which are intentionally bent by surgical personnel during implantation or that flexible portion 110 might be curved due to the relative positions and orientations of vertebrae 22 and 22′.

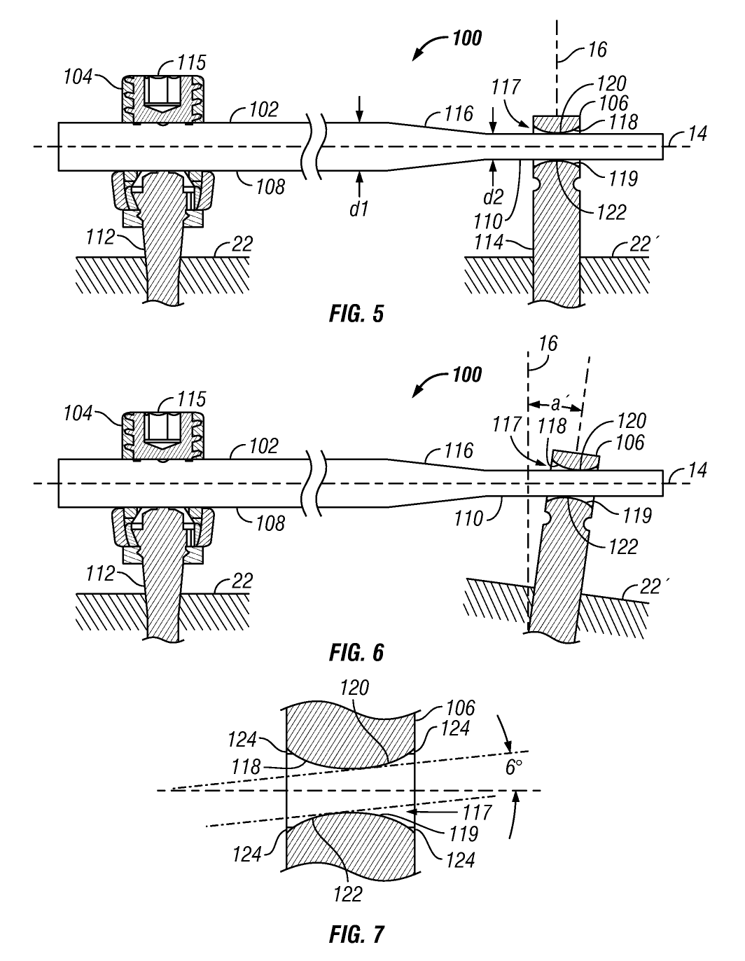

Now with reference to FIG. 5, a cross sectional view of one embodiment of spinal stabilization system 100 is illustrated. More specifically, FIG. 5 illustrates cranial-caudal axis 14, anterior-posterior axis 16, vertebrae 22, 22′, spinal stabilization system 100, spinal stabilization rod 102, clamping collar 104, pivoting collar 106, rigid portion 108 of spinal stabilization rod 102, flexible portion 110 of spinal stabilization rod 102, bone screws 112 and 114, closure member 115, transition portion 116 of spinal stabilization rod 102, bore 117 of pivoting collar 106, internal surfaces 118 and 119 and points of contact 120 and 122 of pivoting collar 106.

Among other features of various embodiments, FIG. 5 illustrates that closure member 115 can be used in conjunction with clamping collar 104 to securely clamp rigid portion 108 of spinal stabilization rod 102 in place. FIG. 5 also illustrates that spinal stabilization rod 102 can include transition portion 116 between rigid portion 108 and flexible portion 110. In some embodiments, the strengths of rigid portion 108 and flexible portion 110 can be determined by their respective diameters (or other dimensions, shapes, etc.) particularly when spinal stabilization rod 102 is formed from one continuous material such as polyetheretherketone (PEEK).

With regard to the engagement between spinal stabilization rod 102 and pivoting collar 106, FIG. 5 illustrates that pivoting collar 106 can define a bore 117 or other cavity. Bore 117 can extend through the body of pivoting collar 106 generally in parallel with cranial-caudal axis 14. Bore 117 can further define internal surfaces 118 and 119 which can be shaped and dimensioned to allow spinal stabilization rod 102 (or certain portions thereof) to pivot about contact points 120 and 122 within bore 117. Spinal stabilization rod 102 can also slidably engage internal surfaces 118 and 119. In some embodiments, pivoting collar 106 is made from titanium and internal surfaces 118 and 119 are polished to a finish sufficient to reduce sliding friction between internal surfaces 118 and 119 and spinal stabilization rod 102. Furthermore, spinal stabilization rod 102 can be made of a material such as PEEK which has a low coefficient of friction with the material of pivoting collar 106. Thus, spinal stabilization rod 102 can both translate and pivot relative to pivoting collar 106 thereby allowing vertebra 22′ to translate and rotate relative to vertebra 22. By allowing vertebrae 22′ and 22 to translate and rotate relative to each other, vertebrae 22′ and 22 can rotate about their natural centers of rotation. Thus, loads imposed on, and generated by, spine 20 (see FIG. 2) and spinal stabilization system 100 can be reduced if not eliminated by spinal stabilization rod 102 and pivoting collar 106.

For instance, a comparison of FIGS. 5 and 6 illustrates that some movement of patient 10 might cause pivoting collar 106 to rotate through angle “a” about medial-lateral axis 12 and away from anterior-posterior axis 16. As pivoting collar 106 rotates through angle “a”, spinal stabilization rod 102 can slide along internal surfaces 118 and 119. Additionally, points of contact 120 and 122 can move as a result of the kinematic interaction between spinal stabilization rod 102 and pivoting collar 106. For instance, when pivoting collar 106 rotates clockwise (as shown in FIGS. 5 and 6), upper contact point 120 can move to the right while lower contact point 122 can move to the left. Should pivoting collar 106 rotate counterclockwise, upper contact point 120 can move to the left while lower contact point 122 can move to the right. No matter which direction pivoting collar 106 rotates, spinal stabilization rod 102 can pivot about contact points 120 and 122 within bore 117.

FIGS. 5 and 6 also illustrate flexible portion 110 of spinal stabilization rod 102. Flexible portion 110 can be made of the same material as rigid portion 108 of spinal stabilization rod 102. In some embodiments, spinal stabilization rod 102 can include transition portion 116 between rigid portion 108 and flexible portion 110. In some embodiments, flexible portion 110 can be similar to rigid portion 108 except perhaps having different, and smaller, dimensions. For instance, spinal stabilization rod 102 can have a generally circular cross section and rigid portion 108 can have diameter “d1” while flexible portion 110 can have another and smaller dimension “d2.” Thus, flexible portion 110 can be more flexible than rigid portion 108. In some embodiments, the smaller dimensions of flexible portion 110 can facilitate the pivoting of spinal stabilization rod 102 in bore 117 of pivoting collar 106.

With reference now to FIG. 7, internal surfaces 118 and 119 of pivoting collar 106 can be shaped and dimensioned so that internal surfaces 118 and 119 limit the range of motion of spinal stabilization rod 102. In some embodiments, internal surfaces 118 and 119 can define lips 124 around the outer periphery of bore 117. Lips 124 can be raised relative to the general contour of internal surfaces 118 and 119. Thus, as spinal stabilization rod 102 pivots it comes into contact with at least one lip 124 and is therefore constrained from further motion. Moreover, it can be the case that spinal stabilization rod 102 comes into contact with one lip 124 on upper internal surface 118 and another lip 124 on lower surface 119 and on the opposite side of bore 117. In some embodiments, lips 124 are shaped, dimensioned, and position to limit the range of motion of spinal stabilization rod 102 to the same limit (e.g., 6 degrees). In some embodiments, each lip 124 can be shaped, dimensioned, and positioned to limit the range of motion of spinal stabilization rod 102 to selected values.

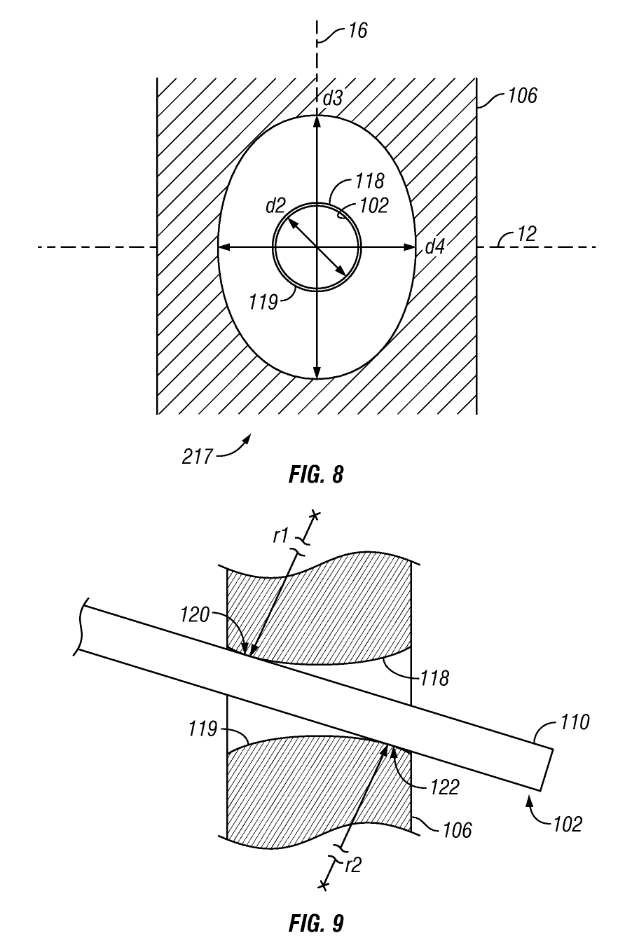

With reference now to FIG. 8, in some embodiments, bore 217 of pivoting collar 106 can be shaped and dimensioned to limit the range of motion of spinal stabilization rod 102 to the same limit in all directions. In some embodiments, however, bore 217 can be shaped and dimensioned to provide selected limits to the range of motion of spinal stabilization rod 102 in differing directions. More specifically, bore 217 can have an oval cross sectional shape as shown with major diameter d3 and minor diameter d4. Furthermore, bore 117 can define lateral surfaces and corresponding contact points thereon. Thus, spinal stabilization rod 102 can have one range of motion (defined by diameter d3) to pivot about medial-lateral axis 12 and another range of motion (defined by diameter d4) to pivot about anterior-posterior axis 16. In some embodiments, Diameter d3 or d4 can correspond to diameter d2 of flexible portion 110 of spinal stabilization rod 102. Thus, pivoting collar 106 can provide some range of motion in one direction while limiting spinal stabilization rod 102 to less or no motion in another direction.

With reference now to FIG. 9, one embodiment of spinal stabilization system 100 is shown. More particularly, flexible portion 110 is shown engaged with pivoting collar 106. FIG. 9 further illustrates that upper internal surface 118 can have radius of curvature r1 and lower internal surface 119 can have radius of curvature r2. Flexible portion 110 of spinal stabilization rod 102 is shown in FIG. 9 as having pivoted about contact points 120 and 122 up to the range of motion permitted by internal surfaces 118 and 119. Moreover, flexible portion 110 of spinal stabilization rod 102 is also shown as having flexed around internal surfaces 118 and 119 so that it follows radii of curvature r1 and r2 for at least some portion of its length. Because spinal stabilization rod 102 has flexed to some degree, spinal stabilization rod 102 can act as a spring and resist further motion. The amount of resistance of spinal stabilization rod 102 to further movement can be tailored by selected the material, shape, dimensions, etc. of flexible portion 110 to provide a desired spring constant. Thus, the resistance to movement provided by flexible portion 110 can change (e.g., increase or decrease) linearly (or otherwise) with that motion.

To attach embodiments of spinal stabilization system 100 to spine 20, surgical personnel can prepare patient 10 for surgery and open an incision generally near spine 20. In some embodiments, surgical personnel can use a posterior approach to spine 20 to attach spinal stabilization system 100 to spine 20. Surgical personnel can attach one or more clamping collars 104 to selected vertebrae 22. Surgical personnel can also attach one or more pivoting collars 106 to other selected vertebrae 22. Surgical personnel may then engage pivoting collar 106 with flexible portion 110 of spinal stabilization rod 102. More specifically, surgical personnel can insert flexible portion 110 through bore 117 of pivoting collar 106.

Surgical personnel can place rigid portion 108 of spinal stabilization rod 102 in, or near, clamping collar 104. If desired, surgical personnel can reduce spinal stabilization rod 102 into clamping collar 104. With spinal stabilization rod 102 seated in clamping collar 104, surgical personnel can advance closure member 115 (see FIGS. 4 or 5) toward spinal stabilization rod 102. Closure member 115 can clamp spinal stabilization rod 102 against the seat of clamping collar 104 thereby locking spinal stabilization rod 102 in place in clamping collar 104. Surgical personnel can evaluate spinal stabilization system 100 and make adjustments as desired before closing the incision.

Thus, patients 10 treated with spinal stabilization system 100 (see FIG. 1) may flex and extend their backs. Patients 10 may also rotate their torsos and bend side-to-side. Accordingly, patients 10 may enjoy greater ranges of motion while experiencing less discomfort than previously possible. Spinal stabilization systems 100 and, more particularly, pivoting collars 106 can be smaller and therefore less intrusive than clamping collars 104. Spinal stabilization systems 100 of various embodiments can be simpler and have fewer parts (e.g., closure member 115) than previously available spinal stabilization systems.

Although embodiments have been described in detail herein, it should be understood that the description is by way of example only and is not to be construed in a limiting sense. It is to be further understood, therefore, that numerous changes in the details of the embodiments and additional embodiments will be apparent, and may be made by, persons of ordinary skill in the art having reference to this description. It is contemplated that all such changes and additional embodiments are within scope of the claims below and their legal equivalents.

What is claimed is:

1. A system for dynamically stabilizing a portion of a spine, the system comprising:
a spinal stabilization rod having a flexible portion; and
a pivoting collar having a bore through which the flexible portion of the spinal stabilization rod is acceptable, wherein the bore of the pivoting collar having a superior internal surface having a first curvature and an inferior internal surface having a second curvature, wherein the first curvature of the superior internal surface and the second curvature of the inferior Internal surface define range of motion of the spinal stabilization rod, and wherein the spinal stabilization rod pivots about a contact point within the bore of the pivoting collar.

2. The system of claim 1, wherein the contact point is a superior contact point on the superior internal surface of the bore of the pivoting collar or an inferior contact point on the inferior internal surface of the bore of the pivoting collar.

3. The system of claim 1, wherein the superior internal surface and the inferior internal surface of the bore define lips around outer periphery of the bore.

4. The system of claim 3, wherein the lips are structured to constrain motion of the spinal stabilization rod.

5. The system of claim 4, wherein the lips are structured to constrain motion of the spinal stabilization rod in all directions.

6. The system of claim 4, wherein the lips are structured to constrain different degrees of motion of the spinal stabilization rod in differing directions.

7. The system of claim 1, wherein the range of motion is about ± six degrees.

8. The system of claim 1, wherein the bore further defines a lateral surface, wherein the spinal stabilization rod pivots about a medial-lateral axis and an anterior-posterior axis within the bore of the pivoting collar.

9. The system of claim 1, wherein at least a portion of the bore has an oval cross sectional shape in a plane parallel to a longitudinal axis of the bore.

10. The system of claim 1, further comprising a clamping collar wherein the clamping collar has an opening for accepting a rigid portion of the spinal stabilization rod.

11. The system of claim 10, wherein the flexible portion of the spinal stabilization rod has a first diameter that is smaller than a second diameter of the rigid portion of the spinal stabilization rod.

12. The system of claim 11, wherein the spinal stabilization rod is formed from one continuous material and wherein the spinal stabilization rod further comprises a transition portion between the flexible portion of the spinal stabilization rod and the rigid portion of the spinal stabilization rod.

13. The system of claim 1, wherein the spinal stabilization rod is at least partially elastically bendable about the contact point.

14. The system of claim 1, wherein the pivoting collar is a part of a bone fastener.

15. A pivoting collar for spinal stabilization, comprising:
a body and a bore through the body, wherein the bore has a superior internal surface having a first curvature, an inferior internal surface having a second curvature, and a lateral internal surface, wherein the first curvature of the superior internal surface, the second curvature of the inferior internal surface, and the lateral internal surface define range of motion of a spinal stabilization rod which is coupled to the pivoting collar through the bore and which pivots about a medial-lateral axis and an anterior-posterior axis within the bore of the pivoting collar.

16. The pivoting collar of claim 15, further comprising lips defined by the superior internal surface and the inferior internal surface of the bore around outer periphery of the bore to constrain motion of the spinal stabilization rod.

17. The pivoting collar of claim 16, wherein the lips are structured to constrain the spinal stabilization rod to about six degrees of motion in all directions.

18. The pivoting collar of claim 16, wherein the lips are structured to constrain different degrees of motion of the spinal stabilization rod in differing directions.

19. The pivoting collar of claim 15, wherein at least a portion of the bore has an oval cross sectional shape in a plane parallel to a longitudinal axis of the bore.

20. A system for dynamically stabilizing a portion of a spine, the system comprising:
a dynamic spinal stabilization rod having a first portion, a second portion, and a transition portion between the first portion and the second portion;
a pivoting collar having a bore through which the first portion of the dynamic spinal stabilization rod is acceptable, wherein the bore of the pivoting collar having a superior internal surface having a first curvature and an inferior internal surface having a second curvature, wherein the first curvature of the superior internal surface and the second curvature of the inferior internal surface define range of motion of the dynamic spinal stabilization rod, and wherein the dynamic spinal stabilization rod pivots about a contact point within the bore of the pivoting collar; and
a clamping collar having an opening for accepting the second portion of the dynamic spinal stabilization rod, wherein the first portion of the dynamic spinal stabilization rod has a first diameter that is smaller than a second diameter of the second portion of the dynamic spinal stabilization rod, wherein the dynamic spinal stabilization rod is at least partially elastically bendable about the contact point within the bore of the pivoting collar.
